(19) United States
(12) Patent Application Publication
(10) Pub. No.: US 2002/0026657 A1
(43) Pub. Date: Feb. 28, 2002

(54) Genetic control of fruit ripening

(76) Inventors: Colin Roger Bird, Bracknell (GB); Graham Barron Seymour, Wellesbourne (GB); Rosybel De Jesus Medina-Suarez, Brentwood (GB)

Correspondence Address: HALE AND DORR, LLP, 60 STATE STREET, BOSTON, MA, 02109

(21) Appl. No.: 09/949,052

(22) Filed: Sep. 7, 2001

Related U.S. Application Data
(63) Continuation of application No. 09/432,122, filed on Nov. 2, 1999.

(30) Foreign Application Priority Data
May 5, 1998 (GB) PCT/GB98/01297
May 20, 1997 (GB) 9710370.9

(51) Int. Cl. C12N 15/82

(52) U.S. Cl. 800/278

(57) ABSTRACT

A method of modulating the ripening and/or senescence characteristics in plants of the genus Musa comprises transforming plants with one or more sequences obtainable from the deposited cDNA library having the accession number 40183, regenerating said plants and selecting from the population of transformants those plants having modulated and/or tissue senescence characteristics.

This invention relates generally to the modification of a plant phenotype by the regulation of plant gene expression. More specifically it relates to the modulation of the ripening and/or tissue senescence characteristics and plants derived therefrom. Two principal methods for the control of expression are known, viz.: overexpression and underexpression. Overexpression is achieved by insertion of one or more than one extra copies of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation, “cosuppression” or “gene silencing”. Both of these methods lead to an inhibition of expression of the target gene. Other lesser used methods involve modification of the genetic control elements, the promoter and control sequences, to achieve greater or lesser expression of an inserted gene.

There is no reason to doubt the operability of sense/cosuppression technology. It is well established, used routinely in laboratories around the world and products in which it is used are on the market.

Gene control by any of these methods requires the insertion of the most favoured gene or genes into plant material which can be regenerated into plants. This transformation process can be performed via a number of methods, for example: the agrobacterium-mediated transformation method.

In the microparticle bombardment method. microparticles of dense material, usually gold or tungsten, are fired at high velocity at the target cells where they penetrate the cells, opening an aperture in the cell wall through which DNA may enter. The DNA may be coated on to the microparticles or may be added to the culture medium. In microinjection, the DNA is inserted by injection into individual cells via an ultrafine hollow needle.

Another method, viz. fibre-mediated transformation, applicable to both monocots and dicots, involves creating a suspension of the target cells in a liquid, adding microscopic needle-like material, such as silicon carbide or silicon nitride “whiskers”, and agitating so that the cells and whiskers collide and DNA present in the liquid enters the cell.

In summary then, the requirements for both sense and antisense technology are known and the methods by which the required sequences may be introduced are known. What remains then is to identify genes whose regulation will be expected to have a desired effect, isolate them or isolate a fragment of sufficiently effective length, construct a chimeric gene in which the effective fragment is inserted between promoter and termination signals, and insert the construct into cells of the target plant species by transformation. Whole plants may then be regenerated from the transformed cells.

One suitable application of the present invention is the modulation of ripening and/or senescence processes in banana.

Bananas are a globally important fruit crop. They are not only a popular dessert fruit, but represent a vital carbohydrate staple in the tropics with as many as 100 million people subsisting on bananas and plantains as their main energy source. The cultivated dessert banana is commonly triploid, parthenocarpic and belongs to the musa AAA genome group, eg. Cavendish subtypes. Bananas are climacteric fruits and ripening is regulated by ethylene produced by the fruit and involves numerous biochemical changes including the conversion of starch to sugars, cell wall disassembly, synthesis of volatile compounds, changes in phenolic constituents and degradation of chlorophyll in the peel. The conversion of starch to sugars is particularly striking, where starch accounts for 20-25% of the fresh weight of the unripe fruit and depending on the genetic background, can be converted almost entirely to sugars. The triploid nature of the cultivated dessert banana crop has hampered conventional methods of breeding for improved characteristics. As a result of this an enormous pool of genetic resources for enhancing postharvest characteristics of the fruit has remained untapped.

According to the present invention there is provided a method of modulating the fruit ripening or tissue senescence characteristics of a plant of the genus Musa comprising inserting into the genome of said plant a DNA construct comprising in sequence a promoter region which is operable in plant cells, a DNA having a nucleotide sequence selected from SEQ ID Nos. 1-73, complementary sequences of SEQ ID Nos. 1-73 and variants of said sequences permitted by degeneracy of the genetic code and a transcription termination sequence, and selecting from the population of regenerants those transformants with modulated fruit ripening or tissue senescence characteristics.

The invention also provides a method as described above wherein the said DNA insert comprises a full length polynucleotide coding sequence which includes a polynucleotide sequence as shown in any one of SEQ ID Nos. 1-73. The promoter of the said DNA construct may be constitutive, developmentally regulated, or switchable. It may additionally be tissue specific or organ specific. The promoter may specifically be either the SAG 1 promoter, the polyubiquitin promoter or the banana ACC oxidase promoter.

Suitable transformation methods for use with the present invention include the Agrobacterium, microparticle bombardment fibre mediated or direct insertion methods.

The invention further provides plant material, plants, their progeny and seed produced according to a method as described above characterised in that said plant material and plants exhibit modulated ripening or tissue senescence characteristics.

The gene sequences of the present invention may be synthesised ab initio, using the sequence data provided in the sequence listing provided herewith, or isolated from a library using the standard techniques know within the art. To assist the isolation of these polynucleotides we have deposited with the National Collection of Industrial & Marine Bacteria, St. Machar Drive, Aberdeen, UK, a cDNA library of the banana peel ripening related genes. The library was deposited on Jul. 9, 1996 and has the Accession Number 40813.

Thus, this invention is based on the identification of genes which encode proteins involved in banana ripening-related processes, specifically within banana peel. The DNA sequences may be used in the process of modifying the plant ripening characteristics of plants and/or fruit.

By virtue of this invention banana plants can be generated which, amongst other phenotypic modifications, may have one or more of the following fruit characteristics: improved resistance to damage during harvest, packaging and transportation due to slowing of the ripening and over-ripening processes; longer shelf life and better storage characteristics due to reduced activity of degradative pathways (e.g. cell wall hydrolysis); improved processing characteristics due to changed activity of proteins/enzymes contributing to factors such as: viscosity, solids, pH, elasticity; improved flavour and aroma at the point of sale due to modification of the sugar/acid balance and other flavour and aroma components responsible for characteristics of the ripe fruit; modified colour due to changes in activity of enzymes involved in the pathways of pigment biosynthesis (e.g. lycopene, β-carotene, chalcones and anthocyanins); increased resistance to post-harvest pathogens such as fungi. The activity of the ripening-related proteins may be either increased or reduced depending on the characteristics desired for the modified plant part (fruit, leaf, flower, etc). The levels of protein may be increased; for example, by incorporation of additional genes. The additional genes may be designed to give either the same or different spatial and temporal patterns of expression in the fruit. “Antisense” or “partial sense” or other techniques may be used to reduce the expression of ripening-related protein.

The activity of each ripening-related protein or enzyme may be modified either individually or in combination with modification of the activity of one or more other ripening-related proteins/enzymes. In addition, the activities of the ripening-related proteins/enzymes may be modified in combination with modification of the activity of other enzymes involved in fruit ripening or related processes.

DNA constructs according to the invention may comprise a base sequence at least 10 bases (preferably at least 35 bases) in length for transcription into RNA. There is no theoretical upper limit to the base sequence—it may be as long as the relevant mRNA produced by the cell—but for convenience it will generally be found suitable to use sequences between 100 and 1000 bases in length. The preparation of such constructs is described in more detail below.

As a source of the DNA base sequence for transcription, a suitable cDNA or genomic DNA or synthetic polynucleotide may be used. The isolation of suitable ripening-related sequences is described above; it is convenient to use DNA sequences derived from the ripening-related clones deposited at NCIMB in Aberdeen. Sequences coding for the whole, or substantially the whole, of the appropriate ripening-related protein may thus be obtained. Suitable lengths of this DNA sequence may be cut out for use by means of restriction enzymes. When using genomic DNA as the source of a base sequence for transcription it is possible to use either intron or exon regions or a combination of both.

To obtain constructs suitable for expression of the appropriate ripening-related sequence in plant cells, the cDNA sequence as found in one of the banana plasmids or the gene sequence as found in the chromosome of the banana plant may be used. Recombinant DNA constructs may be made using standard techniques. In this specification “modulation” means either an increase or decrease. More specifically “modulation the ripening or tissue senescence process in plants” means an alteration being either an increase or decrease in the said processes relative to an untreated or transformed plant.

“Ripening process of plants” means the process of maturing or developing. “Senescence” means the progressive deterioration in function of cells, tissues, organs etc., related to the period of time since that function commenced.

“Plant material” includes plant cells and any other type of plant regenerable material. “Full length polynucleotide coding sequence” includes a polynucleotide coding for the whole or substantially the whole of the appropriate ripening related mRNA/protein. The invention will now be described by way of an example where:

TABLE 1. Is a list of all the clones isolated from banana peel and the corresponding sequence identity number as provided in the sequence listing herein. The table also illustrates the approximate clone size, the percentage identity and nucleotide similarity based on the results obtained from comparisons with the EMBL sequence database. Therefore, the table provides the putative gene identity based on these comparisons, corresponding published sequences and their database accession numbers.

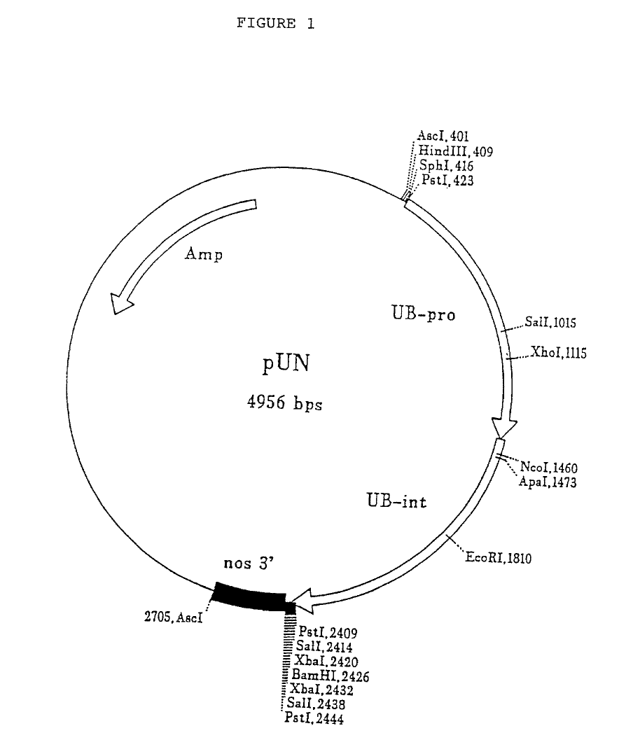

FIG. 1. Plant transformation vector pUN, containing the UBI polyubiquitin promoter.

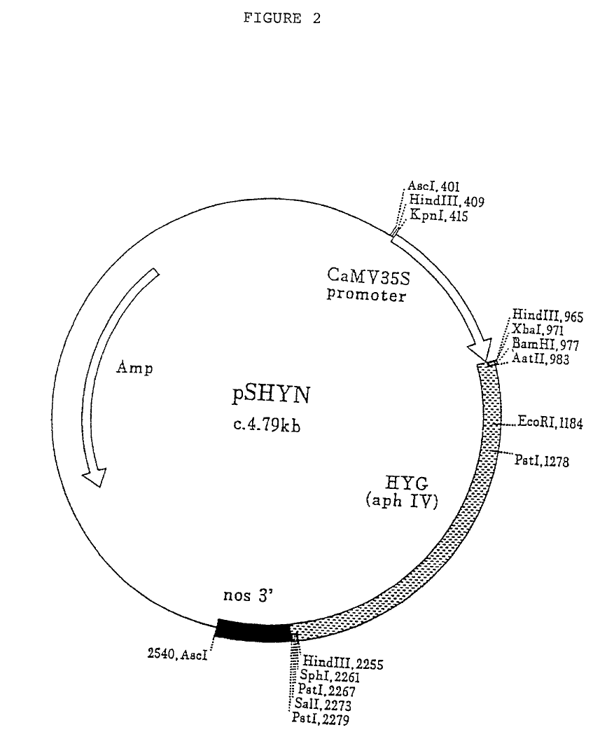

FIG. 2. Plant transformation vector pSHYN, containing hygromycin resistance gene for selection of transformed plants.

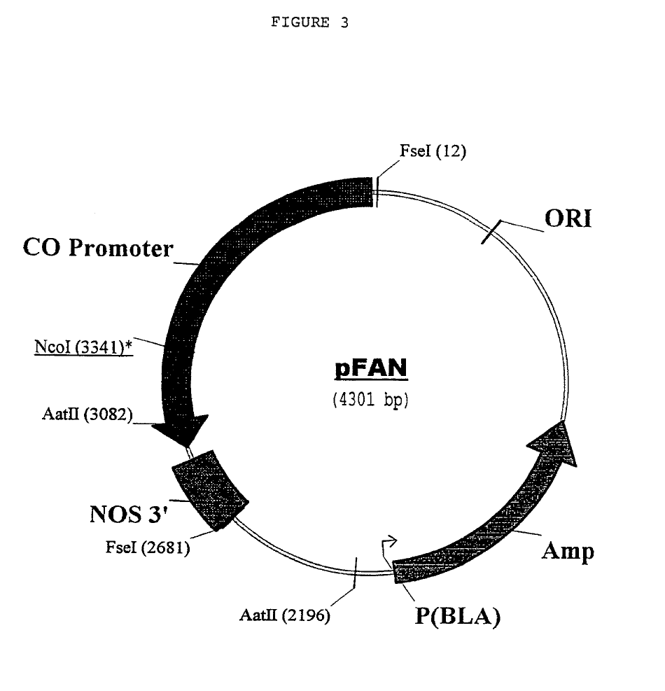

FIG. 3. Plant transformation vector pFAN, containing the banana ACC oxidase promoter.

### EXAMPLE 1

Construction of a cDNA Library of Ripening Genes

1.1 Isolation of Messenger RNA

Total RNA was isolated from ripening (24 hours after ethylene treatment) banana peel (Musa acuminata cv. Grand Nain) as described by Chang et al, Plant Molecular Biology Reporter, Vol. 11(2) 113-116 (1993). Messenger RNA was isolated from total RNA by Oligo(dT)-cellulose chromatography according to Bantle et al., Analytical Biochemistry 72, 413-427 (1976).

1.2 Synthesis of cDNA and Cloning into Vector

The first and second strands of the cDNAs were synthesised from the messenger RNAs using a commercial cDNA synthesis kit (Catalog No. 200450, ZAP Express™ Gold Cloning kit, Stratagene Ltd, Cambridge, Cambs, UK). Double stranded cDNAs were cloned into the ZAP Express™ vector, packaged, mixed with plating bacteria to determine titre and for library screening, following instructions of the suppliers protocol.

1.3 Screening of the cDNA Library From Banana Peel.

The unamplified cDNA library from ripening banana peel was differentially screened using cDNA from unripe and ripening banana peel tissue. A proportion of the library was plated individually at low density and duplicate plaque lifts made onto Hybond N nylon filters (Amersham) according to the manufacturer's instructions. One filter was hybridised to dCTP radiolabeled cDNA from green fruit and the duplicate filter hybridised to dCTP radiolabeled cDNA from ripening fruit. Hybridisations were at high stringency. Plaques hybridising preferentially with ripening or green radiolabeled cDNA were picked and replated for a second round of selection by differential screening. These clones were numbered as ripening up- or down-regulated peel clones. The clones were in-vivo excised from the ZAP express™ vector into the pBK-CMV phagemid vector using the ExAssist™ interference-resistant helper phage, following instructions from manufacturers protocol.

1.4 Characterisation of the Ripening Peel cDNA Library and the Ripening-Related Clones.

The ripening cDNA library from peel tissue were prepared with an efficiency of 3.2×10⁵ plaque-forming units per microgram of cDNA. The sizes of the inserts in the peel library was 0.4-6.7 Kb with a mean size insert of 1.47 Kb.

From the 250 plaques used in the first screen, 73 putative ripening-related clones were obtained. These 73 clones were partially sequenced using the ABI PRISMTM Dye Terminator Cycle Sequencing Ready Reaction kit with AmpliTaq® DNA polymerise (Applied Biosystems, Warrington, Cheshire, UK) with forward primers specific for the pBK-CMV vector. From these, the following ripening-related clones were selected. Comparisons of this sequences in the EMBL database using GCG (‘Wisconsing’) software has identified homologies for the clones listed in TABLE 1 below.

### EXAMPLE 2

Construction of partial sense RNA vectors with the maize polyubiquitin promoter. A vector is constructed using the sequences corresponding to a fragment of the inserts of one of the sequences 1 to 73. This fragment is synthesised by polymerase chain reaction using synthetic primers incorporating BamHI restriction sites suitable for cloning between a maize UBI polyubiquitin promoter (Christensen et al, 1992, Plant Molecular Biology, 18:675-689) and a nopaline synthase 3′ end termination sequences in the vector pUN (FIG. 1.).

The partial sense expression cassette is excised by digestion with AscI, the ends of the fragment are made flush with T4 polymerase and it is cloned into the vector pSHYN (FIG. 2.) which has been cut with KpnI and the ends made flush with Klenow polymerase. pSHYN contains hygromycin resistance gene for selection of transformed plants.

After synthesis of the vector, the structure and orientation of the sequences are confirmed by DNA sequence analysis.

### EXAMPLE 3

Construction of partial sense RNA vectors with a fruit enhanced promoter.

The 1386 bp HindIII fragment containing the banana ACC oxidase promoter (PCT Application No. WO97/38106) is cloned the HindIII site of a multiple cloning vector to give the vector pFAN.

A vector is constructed using the sequences corresponding to a fragment of the inserts of one of the sequences 1 to 73. This fragment is synthesised by polymerase chain reaction using synthetic primers incorporating BamHI restriction sites suitable for cloning between a maize UBI polyubiquitin promoter (Christensen et al, 1992, Plant Molecular Biology, 18:675-689) and a nopaline synthase 3′ end termination sequences in the vector pFAN (FIG. 3.)

The truncated sense expression cassette is excised by digestion with AscI, the ends of the fragment are made flush with T4 polymerase and it is cloned into the vector pSHYN (FIG. 2.) which has been cut with KpnI and the ends made flush with Klenow polymerase. pSHYN contains hygromycin resistance gene for selection of transformed plants.

After synthesis of the vector, the structure and orientation of the sequences are confirmed by DNA sequence analysis.

### EXAMPLE 4

Construction of an over-expression vector with the maize polyubiquitin promoter. The complete sequence of a ripening related cDNA containing a full open-reading frame is inserted into the vectors described in EXAMPLE 2.

### EXAMPLE 5

Construction of an over-expression vector with a fruit enhanced promoter. The complete sequence of a ripening related cDNA containing a full open reading frame is inserted into the vectors described in EXAMPLE 3.

### EXAMPLE 6

Generation of transformed Musa plants.

Transformed Musa plants containing the vectors are produced by the method described in Sagi et al. (1995) Biotechnology. Vol. 13 pp 481-485. Regenerated transformed plants are identified by their ability to grow on hygromycin and grown to maturity. Ripening fruit are analysed for a modulation in their ripening related or senescence characteristics.

Other suitable transformation methods for banana are described in Sagi et al. (1994) Plant Cell Reports. Vol. 13. pp 262-266. and May et al. (1995) Biotechnology. Vol. 13 pp 486-492.

TABLE 1

| Sequence | | Clone | Size | Gene | | Sequence | |
|---|---|---|---|---|---|---|---|
| Identity | Group | no. | Kb | Identity | % Identity | Bp | Published Sequences |
| SEQ-ID-NO-1 | Peel | 7 | 0.6, 0.4 | Aminocyclopropane | 86.5 | 415 | Musa acuminata X91076 |
| | Upregulated | | | carboxylic oxidase | | | |
| SEQ-ID-NO-2 | Peel | 13 | 0.7, 0.5 | Aminocyclopropane | 94.7 | 152 | Musa acuminata X91076 |
| | Upregulated | | | carboxylic oxidase | | | |
| SEQ-ID-NO-3 | Peel | 23 | 0.8 | Aminocyclopropane | 99.6 | 227 | Musa acuminata X91076 |
| | Upregulated | | | carboxylic oxidase | | | |
| SEQ-ID-NO-4 | Peel | 105 | 0.7, 0.5 | Aminocyclopropane | 99.6 | 227 | Musa acuminata X91076 |
| | Upregulated | | | carboxylic oxidase | | | |
| SEQ-ID-NO-5 | Peel | 8 | 1.9 | Aconitase | 76 | 815 | Cucurbita melo X82840 |
| | Upregulated | | | | | | |
| SEQ-ID-NO-6 | Peel | 11 | 1.7 | Pectate Lyase II | 64.6 | 579 | Zea mays L20140 |
| | Upregulated | | | | | | |
| SEQ-ID-NO-7 | Peel | 12 | 1.8 | Pectate Lyase I | 58 | 276 | Nicotiana tabacum X61102 |
| | Upregulated | | | | | | |
| SEQ-ID-NO-8 | Peel | 22 | 1.8 | Pectate Lyase I | 61.2 | 389 | Lilium longiflorum L18911 |
| | Upregulated | | | | | | |
| SEQ-ID-NO-9 | Peel | 31 | 1.7 | Pectate Lyase II | 64.7 | 546 | Zea mays L20140 |
| | Upregulated | | | | | | |
| SEQ-ID-NO-10 | Peel | 51 | 1.7 | Pectate Lyase II | 66.3 | 661 | Zea mays L20140 |
| | Upregulated | | | | | | |
| SEQ-ID-NO-11 | Peel | 52 | 1.9 | Pectate Lyase I | 59.8 | 361 | Lilium longiflorum L18911 |
| | Upregulated | | | | | | |
| SEQ-ID-NO-12 | Peel | 57 | 1.8 | Pectate Lyase II | 61.9 | 491 | Zea mays L20140 |
| | Upregulated | | | | | | |
| SEQ-ID-NO-13 | Peel | 59 | 1.5 | Pectate Lyase II | 64.6 | 582 | Zea mays L20140 |
| | Upregulated | | | | | | |
| SEQ-ID-NO-14 | Peel | 68 | 6.7 | Pectate Lyase II | 68.2 | 352 | Zea mays L20140 |
| | Upregulated | | | | | | |
| SEQ-ID-NO-15 | Peel | 69 | 1.5 | Pectate Lyase II | 64.3 | 649 | Zea mays L20140 |
| | Upregulated | | | | | | |
| SEQ-ID-NO-16 | Peel | 85 | 1.5 | Pectate Lyase II | 64.2 | 584 | Zea mays L20140 |
| | Upregulated | | | | | | |
| SEQ-ID-NO-17 | Peel | 101 | 1.5 | Pectate Lyase II | 65.1 | 578 | Zea mays L20140 |
| | Upregulated | | | | | | |
| SEQ-ID-NO-18 | Peel | 113 | 1.8 | Pectate Lyase I | 56.4 | 557 | Lilium longiflorum L18911 |
| | Upregulated | | | | | | |
| SEQ-ID-NO-19 | Peel | 114 | 1.7 | Pectate Lyase I | 59.2 | 419 | Licopersicon esculentum |
| | Upregulated | | | | | | X55193 |
| SEQ-ID-NO-20 | Peel | 130 | 1.6 | Pectate Lyase II | 65.3 | 588 | Zea mays L20140 |
| | Upregulated | | | | | | |
| SEQ-ID-NO-21 | Peel | 139 | 1.7 | Pectate Lyase I | 55.1 | 535 | Lilium longiflorum L18911 |
| | Upregulated | | | | | | |
| SEQ-ID-NO-22 | Peel | 16 | 1.1 | Endochitinase | 73.6 | 671 | Oriza sativa X56063 |
| | Upregulated | | | | | | |
| SEQ-ID-NO-23 | Peel | 19 | 1.1 | Endochitinase | 71.6 | 690 | Oriza sativa X56063 |
| | Upregulated | | | | | | |
| SEQ-ID-NO-24 | Peel | 48 | 1 | Endochitinase | 71.1 | 774 | Oriza sativa D16221 |
| | Upregulated | | | | | | |
| SEQ-ID-NO-25 | Peel | 54 | 1.1 | Endochitinase | 69.7 | 634 | Oriza sativa D16221 |
| | Upregulated | | | | | | |
| SEQ-ID-NO-26 | Peel | 91 | 1.2 | Endochitinase | 68.1 | 740 | Oriza sativa D16221 |
| | Upregulated | | | | | | |
| SEQ-ID-NO-27 | Peel | 97 | 1.1 | Endochitinase | 68.5 | 731 | Oriza sativa X56063 |
| | Upregulated | | | | | | |
| SEQ-ID-NO-28 | Peel | 20 | 0.7 | Beta-1,3-Glucanase | 61.9 | 754 | Hordeum vulgare M96939 |
| | Upregulated | | | | | | |
| SEQ-ID-NO-29 | Peel | 33 | 1.2 | Beta-1,3-Glucanase | 60.1 | 697 | Barley M91814 |
| | Upregulated | | | | | | |
| SEQ-ID-NO-30 | Peel | 36 | 1.2 | Beta-1,3-Glucanase | 61.4 | 720 | Barley M91814 |
| | Upregulated | | | | | | |
| SEQ-ID-NO-31 | Peel | 53 | 1.2 | Beta-1,3-Glucanase | 57.3 | 592 | Nicotiana plumbaginifolia |
| | Upregulated | | | | | | M63634 |
| SEQ-ID-NO-32 | Peel | 58 | 1.3 | Beta-1,3-Glucanase | 59.8 | 716 | Hordeum vulgare M96939 |
| | Upregulated | | | | | | |
| SEQ-ID-NO-33 | Peel | 72 | 0.8 | Beta-(1,3:1,4)-D- | 62.7 | 585 | Barley X52572 |
| | Upregulated | | | Glucanase | | | |
| SEQ-ID-NO-34 | Peel | 86 | 1.2 | Beta-1,3-Glucanase | 58.9 | 638 | Hordeum vulgare M96939 |
| | Upregulated | | | | | | |
| SEQ-ID-NO-35 | Peel | 96 | 1.1 | Beta-1,3-Glucanase | 61 | 703 | Hordeum vulgare M96939 |
| | Upregulated | | | | | | |
| SEQ-ID-NO-36 | Peel | 100 | 1.1 | Beta-glucanase | 59.5 | 639 | Nicotiana plumbaginifolia |
| | Upregulated | | | | | | M23120 |
| SEQ-ID-NO-37 | Peel | 102 | 1.1 | Beta-1,3-Glucanase | 59.8 | 487 | Nicotiana plumbaginifolia |
| | Upregulated | | | | | | X07280 |
| SEQ-ID-NO-38 | Peel | 103 | 1.1 | Beta-1,3-Glucanase | 57.8 | 642 | Glicine max A26451 |
| | Upregulated | | | | | | |
| SEQ-ID-NO-39 | Peel | 140 | 1.1 | Endo-1,3-beta- | 59.4 | 647 | Hordeum vulgare M96939 |
| | Upregulated | | | glucanase | | | |
| SEQ-ID-NO-40 | Peel | 89 | 1.3 | Beta-glucosidase | 62 | 510 | Trifolium repens X56733 |
| | Upregulated | | | | | | |
| SEQ-ID-NO-41 | Peel | 129 | 1.3, | Beta-glucosidase | 59.1 | 643 | Trifolium repens X56733 |
| | Upregulated | | 0.6 | | | | |
| SEQ-ID-NO-42 | Peel | 24 | 0.6, | UDP glucose | 74.8 | 785 | Solanium tuberosum |
| | Upregulated | | 0.5 | pyrophosphorylase | | | D00667 |
| SEQ-ID-NO-43 | Peel | 26 | 0.5 | Legumin storage | 63.2 | 190 | Calocedrus decurrens |
| | Upregulated | | | protein | | | X95539 |
| SEQ-ID-NO-44 | Peel | 35 | 0.6, | Legumin storage | 63.2 | 190 | Calocedrus decurrens |
| | Upregulated | | 0.5 | protein | | | X95540 |
| SEQ-ID-NO-45 | Peel | 63 | 0.5 | Legumin storage | 51.7 | 526 | Magnolia salicifolia X82465 |
| | Upregulated | | | protein | | | |
| SEQ-ID-NO-46 | Peel | 29 | 1 | Isoflavonoid | 59.3 | 735 | Arabidopsis thailiana |
| | Upregulated | | | Reductase | | | Z49777 |
| SEQ-ID-NO-47 | Peel | 93 | 1 | Isoflavonoid | 63 | 692 | Solanium tuberosum |
| | Upregulated | | | Reductase | | | X92075 |
| SEQ-ID-NO-48 | Peel | 39 | 1. 0.8, | Extensin | 57.3 | 288 | Chlamidomonas reinhardtii |
| | Upregulated | | 0.7, | | | | X16619 |
| | | | 0.5 | | | | |
| SEQ-ID-NO-49 | Peel | 41 | 1.2 | Chitinase | 57.5 | 454 | Oriza sativa U02286 |
| | Upregulated | | | | | | |
| SEQ-ID-NO-50 | Peel | 57 | 3 | PEP carboxylase | 65.5 | 537 | Glicine max D10717 |
| | Upregulated | | | | | | |
| SEQ-ID-NO-51 | Peel | 109 | 0.9 | Beta-1,3-glucanase | 54.3 | 175 | Nicotiana plumbaginifolia |
| | Upregulated | | | regulator gene | | | M63634 |
| SEQ-ID-NO-52 | Peel | 134 | 2.5, | High Mobility Group | 67.3 | 483 | Zea mays X58282 |
| | Upregulated | | 0.6 | protein | | | |
| SEQ-ID-NO-53 | Peel | 37 | 1.1, | Unknown | — | — | — |
| | Upregulated | | 0.7 | | | | |
| SEQ-ID-NO-54 | Peel | 42 | 2.3 | Unknown | — | — | — |
| | Upregulated | | | | | | |
| SEQ-ID-NO-55 | Peel | 47 | 1 | Unknown | — | — | — |
| | Upregulated | | | | | | |
| SEQ-ID-NO-56 | Peel | 48 | 3.7 | Unknown | — | — | — |
| | Upregulated | | | | | | |
| SEQ-ID-NO-57 | Peel | 54 | 1.3, | Unknown | — | — | — |
| | Upregulated | | 0.7 | | | | |
| SEQ-ID-NO-58 | Peel | 66 | 0.8, | Unknown | — | — | — |
| | Upregulated | | 0.7 | | | | |
| SEQ-ID-NO-59 | Peel | 84 | 1.5, | unknown | — | — | — |
| | Upregulated | | 0.6 | | | | |
| SEQ-ID-NO-60 | Peel | 96 | 1.4 | unknown | — | — | — |
| | Upregulated | | | | | | |
| SEQ-ID-NO-61 | Peel | 97 | 1.1 | unknown | — | — | — |
| | Upregulated | | | | | | |
| SEQ-ID-NO-62 | Peel | 98 | 1.8 | unknown | — | — | — |
| | Upregulated | | | | | | |
| SEQ-ID-NO-63 | Peel | 112 | 1, 0.6 | unknown | — | — | — |
| | Upregulated | | | | | | |
| SEQ-ID-NO-64 | Peel | 24 | 3 | Elongation factor | 54.1 | 268 | Porphyra purpurea U08841 |
| | Down | | | EF1-alpha | | | |
| | regulated | | | | | | |
| SEQ-ID-NO-65 | Peel | 28 | 1.3 | Unknown | — | — | — |
| | Down | | | | | | |
| | regulated | | | | | | |
| SEQ-ID-NO-66 | Peel | 86 | 1.7, 0.5 | Elongation Factor 1- | 80.6 | 708 | Hordeum vulgare Z23130 |
| | Down | | | alpha | | | |
| | regulated | | | | | | |
| SEQ-ID-NO-67 | Peel | 38 | 2.5 | Heat Shock Protein | 87.2 | 218 | Oriza sativa X67711 |
| | Down | | | | | | |
| | regulated | | | | | | |
| SEQ-ID-NO-68 | Peel | 88 | 0.9 | Histone H1 | 60.1 | 619 | Zea mays X57077 |
| | Down | | | | | | |
| | regulated | | | | | | |
| SEQ-ID-NO-69 | Peel | 141 | 1.8, | Wali 7 | 66.4 | 432 | Triticum aestivum L28008 |
| | Down | | 0.8 | | | | |
| | regulated | | | | | | |
| SEQ-ID-NO-70 | Peel | 60 | 2.3 | Unknown | — | — | — |
| | Down | | | | | | |
| | regulated | | | | | | |
| SEQ-ID-NO-71 | Peel | 92 | 3.5 | Unknown | — | — | — |
| | Down | | | | | | |
| | regulated | | | | | | |
| SEQ-ID-NO-72 | Peel | 110 | 0.5 | Unknown | — | — | — |
| | Down | | | | | | |
| | regulated | | | | | | |
| SEQ-ID-NO-73 | Peel | 123 | 0.8 | Unknown | — | — | — |
| | Down | | | | | | |
| | regulated | | | | | | |

1. A method of modulating the fruit ripening or tissue senescence characteristics of a plant of the genus Musa comprising inserting into the genome of said plant a DNA construct comprising in sequence a promoter region which is operable in plant cells, a DNA insert having a nucleotide sequence selected from SEQ ID Nos. 1-73, complementary sequences of SEQ ID Nos. 1-73 and variants of said sequences permitted by degeneracy of the genetic code and a transcription termination sequence, and selecting from the population of regenerants those transformants with modulated fruit ripening or tissue senescence characteristics.

2. A method according to claim 1 wherein the said DNA insert comprises a full length polynucleotide coding sequence which includes a polynucleotide sequence as shown in any one of SEQ ID Nos. 1-73.

3. A method according to claim 1 or claim 2 wherein the said DNA construct comprises a promoter which is constitutive, developmentally regulated, or switchable.

4. A method according to claim 3 wherein said promoter is tissue specific or organ specific.

5. A method according to any one of claims 1 to 4 wherein the promoter is either the SAG 1 promoter, the polyubiquitin promoter or the banana ACC oxidase promoter.

6. A method according to any one of claims 1 to 5 wherein plants are transformed using the Agrobacterium, microparticle bombardment, fibre mediated or direct insertion method.

7. Plant material, plants, their progeny and seed produced according to a method as claimed in any one of claims 1 to 6, characterised in that said plant material and plants exhibit modulated ripening or tissue senescence characteristics.
